(19) United States
(12) Patent Application Publication
(10) Pub. No.: US 2005/0131314 A1
(43) Pub. Date: Jun. 16, 2005

(54) Methods and apparatus for the rapid detection of microorganisms collected from infected sites

(76) Inventors: Robert F. Hird, Pleasant Hill, CA (US); Laurie J. Oleksiewicz, Pleasant Hill, CA (US); Mary J. Hird, Pleasant Hill, CA (US)

Correspondence Address: Junrui Yang, 6815 Devon Way, San Jose, CA, 95129, US

(21) Appl. No.: 10/991,036

(22) Filed: Nov. 16, 2004

Related U.S. Application Data
(60) Provisional application No. 60/520,786, filed on Nov. 17, 2003.

(51) Int. Cl. A61B 10/00; C12Q 1/02; C12Q 1/04; A61B 5/00; B65D 81/00; A61F 2/02; C12Q 1/14

(52) U.S. Cl. 600/572; 435/29; 435/34; 435/36

(57) ABSTRACT

Disclosed herein are methods, reagents and apparatus for rapid detection of microorganisms in swab-collected samples collected from testing objects. Testing objects include food surface, and human body sites.

### CROSS REFERENCE TO RELATED APPLICATIONS

This application claims the benefits of the provisional application, No. 60/520,786, entitled “Methods and Apparatus for the Rapid Detection of Microorganisms Collected from Infected Sites”, filed Nov. 17, 2003, which is hereby incorporated in its entirety for all purposes.

### FIELD OF THE INVENTION

The present invention relates to diagnostic methods, reagents and apparatus, and more particularly to methods, reagents and apparatus for the detection of microorganisms collected from infected sites on testing objects.

### BACKGROUND OF THE INVENTION

Rapid detection of specific microorganisms, for example, infectious pathogens, is of interest to the medical community. Especially important to the community is the need to reduce the potential for the medical care providers to become cross infected. A diagnostic apparatus that is entirely self-contained after the sample is obtained, and gives results in a rapid time frame, is an extremely valuable tool.

One common way to detect the presence of a specific microorganism at a particular body site is by applying a swab to that body site and thus collecting a specimen of the discharge therefrom, transferring some of the swab-collected discharge to grow on a culture plate, and then microscopically examining the colonies of microorganisms thus grown on the culture plate. This method is slow and expensive. In general, it must be conducted in a laboratory, and it takes several days; which makes it unsatisfactory for a quick diagnosis, e.g., in a physician's office.

This transferring method is known to be very inefficient. Usually, such swab transfers succeed in transferring about 15% to 20% of the swab-collected discharge to the surface of the culture plate, and even the most efficient (and much more elaborate and time-consuming) extraction procedures transfer less than 50% of the collected discharge.

The detection of the presence of a specific microorganism at such a body site by the self-contained, direct testing of the swab-collected discharge, on the swab, without transferring that discharge to a culture plate, has many advantages. This direct testing method permits the testing of 100% of the collected discharge, and is 20 to 50 times more sensitive than the above-described transferring method. This self-contained diagnostic method greatly reduces the risk of providers for their exposure to the infectious pathogens. It also reduces the testing time significantly.

### SUMMARY OF THE INVENTION

Accordingly, it is an object of the present invention to provide reagents by the use of which the presence of specific microorganisms (infectious agents) in samples collected by swabs from testing objects may be readily detected on the collecting swabs, i.e., without transferring the collected samples to culture plates and the like.

Another object of the present invention is to provide methods of thus directly testing swab-collected samples, which methods are characterized by greatly improved diagnostic sensitivity as compared with the transferring testing methods in which some of the swab-collected samples are transferred to a culture medium.

Yet another object of the present invention is to provide methods of testing swab-collected samples from testing objects which require less specialized labor than said transferring methods and can be carried out much more rapidly.

A further object of the present invention is to provide methods for detecting the presence of specific microorganisms on testing objects, which methods greatly reduce the possibility of contaminating the samples collected because the detection steps thereof are carried out directly on the collecting swabs.

An additional object of the present invention is to provide methods of detecting, by very simple testing steps which can easily and rapidly be carried out, a wide variety of microorganisms. The microorganisms include, but not limited to, Chlamydia trachomatis, Neisseria gonohorrea, Candida albicans (yeast), Group A Streptococcus, Group B Streptococcus, Cryptosporidium, Staphylococcus aureus, Pseudomonas aerginosa, SARS virus and Anthrax.

Yet another object of the present invention is to provide reagents to be applied to swab-collected samples, whereby to reveal the presence of particular microorganisms in the swab-collected samples by the production of fluorometric and/or colorimetric reactions, resulting in a fluorescent or color endpoint easily determined by an ultraviolet light source at a predetermined wavelength, or by inspection with the unaided human eye.

A yet further object of the present invention is to provide diagnostic apparatus which can be used to rapidly detect the presence of particular microorganisms in the swab-collected samples from testing objects, without the necessity for elaborate laboratory facilities.

Yet another further object of the present invention is to provide methods, reagents and apparatus which can be used to quantitatively detect the presence of particular microorganisms in the swab-collected samples from testing objects.

Other objects of the present invention will in part be obvious and will in part appear hereinafter.

The present invention, accordingly, comprises the several steps and the relation of one or more of such steps with respect to each of the others, and the apparatus embodying features of construction, combinations of elements, arrangements of parts, and reagents which are adapted to effect or be used in such steps, all as exemplified in the following disclosure, and the scope of the present invention will be indicated in the appended claims.

One embodiment of the present invention is to provide a method for detecting a microorganism on a testing object. The method comprises the following steps: applying a swab to said testing object to collect on said swab a sample from said testing object; contacting said swab and said sample collected thereon with at least one reagent, wherein said at least one reagent being capable of reacting with said microorganism in said sample to produce a detectable product; and determining from said detectable product, the presence or absence of said microorganism on said testing object. Testing objects include, but not limited to, food surfaces, animal body sites or human body sites. When the testing object is a human body site, the sample being collected can be a human specimen or discharge.

Another embodiment of the present invention is to provide a method which can be used to detect one or more microorganisms. The microorganisms include, but not limited to, Chlamydia trachomatis, Neisseria gonohorrea, Candida albicans (yeast), Group A Streptococcus, Group B Streptococcus, Cryptosporidium, Staphylococcus aureus, Pseudomonas aerginosa, SARS virus or Anthrax.

In accordance with one embodiment of the present invention, one or more reagents are provided which when come into contact with swab-collected samples collected from testing objects produce detectable products indicating the presence of particular microorganisms in said swab-collected samples.

In accordance with another embodiment of the present invention, reagents are provided which indicate the presence of specific microorganisms in said swab-collected samples by producing detectable flourometric or colorimetric reactions.

In accordance with yet another embodiment of the present invention, diagnostic apparatus are provided which can be use to detect the presence of microorganisms.

For a fuller understanding of the nature and objects of the present invention, reference should be had to the following detailed description, taken in connection with the accompanying drawings.

### BRIEF DESCRIPTION OF THE DRAWINGS

FIG. 1 is a perspective view of a diagnostic swabbing and testing apparatus constructed in accordance with the present invention;

FIG. 2 is a sectional view of the diagnostic apparatus of FIG. 1, taken along its major axis;

FIG. 3 is a partial sectional view of the apparatus of FIG. 1 taken during a particular phase of its operation;

FIG. 4 is a perspective view of a modification of the apparatus of FIG. 1 taken during a particular phase of the operation; and

FIG. 5 is an example of a diagnostic apparatus constructed according to one or more embodiments of the present invention.

### DESCRIPTION OF THE INVENTION

Referring now to FIG. 1, there is shown a diagnostic swab and testing device 10 embodying the present invention.

As seen in FIG. 1, diagnostic device 10 is comprised of a swab unit 12 and a swab cover unit 14.

As best seen by comparison of FIGS. 1 and 2, swab unit 12 is comprised of a hollow cylindrical barrel or handle member 16 which is closed at one end by a plug 18. Plug 18 is preferably affixed in the position shown in FIGS. 1 and 2 by cementing.

Barrel 16 contains, in the embodiment of FIGS. 1 through 3, a pair of reagent ampoules 20, 22.

After the insertion of reagent ampoules 20, 22 the end of barrel 16 is closed by a swab shaft assembly 24, comprised of a sleeve 26, a bushing 28, a hollow shaft 30, and a swab tip 32.

As best seen in FIG. 2, sleeve 26 is cemented into the end of barrel 16 opposite plug 18, and bushing 28 is cemented into sleeve 26. Hollow shaft 24 is cemented into the smaller central bore of bushing 28, and swab tip 32 is cemented to the opposite end of shaft 24. Tip 32 is fabricated from a suitable fibrous material which is well adapted to collect specimen material from a body site, and is permeable to materials contained in reagent ampoules 20, 22. Tip 32 is adhered to the end of shaft 24 by well known means.

As also best seen in FIG. 2, cover unit 14 is comprised of an inner tube 34 and outer tube 36. The larger diameter end of tube 34 is adapted to close-fittingly embrace sleeve 26, and is provided with a locking groove 38 which is adapted to receive a ridge 40 raised on the outer surface of sleeve 26, whereby to releasably attach cover unit 14 to swab unit 12 in such manner that cover unit 14 can be removed from swab unit 14 by a user who forcibly separates the two manually, but will otherwise remain in position, covering swab shaft assembly 24 and protecting it from air-borne contaminants.

As further seen in FIG. 2, outer tube 36 close-fittingly embraces one inner tube 34, and is cemented to that end of inner tube 34.

Outer tube 36 contains a reagent ampoule 42 which in the preferred embodiment of the present invention will be called the color developer ampoule or developer ampoule, and is provided at the end opposite swab assembly 12 with a suitable plug 44, which is cemented into the outer end of outer tube 36 to retain color developer ampoule 42 therein.

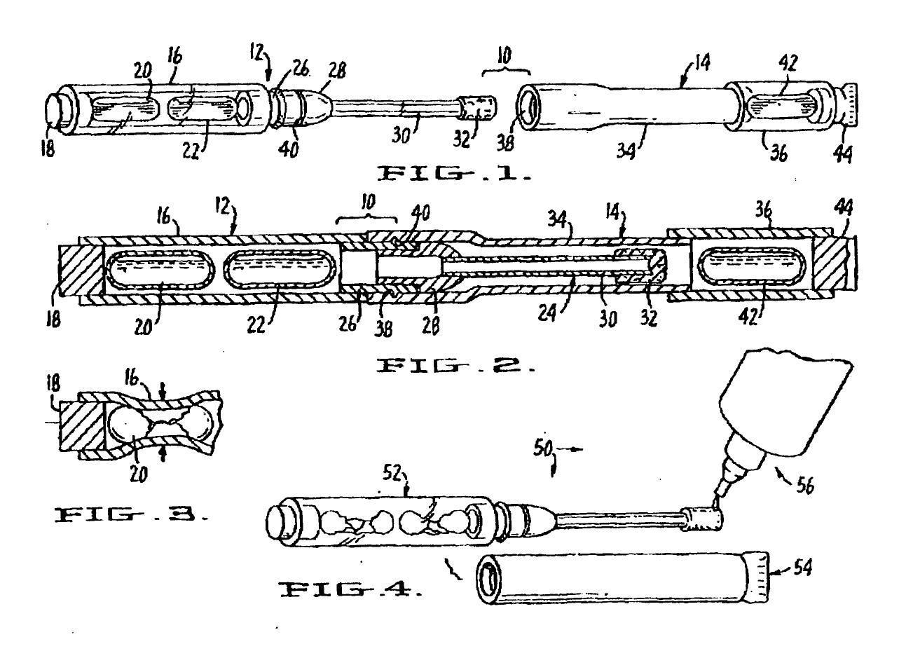

As illustrated in FIG. 3, barrel 16 is non-rigid, and thus may be manually squeezed to crush or rupture reagent ampoule 20, which is fabricated from suitable frangible material, thereby releasing the reagent in ampoule 20 to the interior of barrel 16, whence it can be dispensed by gravity to the interior shaft 30, and thus to permeable tip 32.

It is to be understood that reagent ampoule 22 is also frangible and thus crushable by squeezing barrel 16, after which its contents can be similarly dispensed by gravity to permeable tip 32.

It is further to be noted that outer tube 36 is also non-rigid ant that reagent (color developer) ampoule 42 is also frangible, whereby the contents of reagent ampoule 42 can be applied to swab tip 32 when cover unit 14 is locked in place on swab unit 12.

Referring now to FIG. 4, there is shown a second diagnostic device 50 embodying the present invention.

Diagnostic device 50 comprises a swab unit 52 which may be in all respects substantially identical to swab unit 12 of the embodiment of FIGS. 1 through 3.

Diagnostic device 50 further comprises a cover unit 54 which is similar to that of cover unit 14 of the embodiment of FIGS. 1 through 4 but lacks outer tube 36 and reagent ampoule 42.

Diagnostic device 50 further comprises a dispenser bottle 56 by means of which a developing agent, which may be the same agent as that contained in reagent ampoule 42, can be manually dispensed to the tip of swab unit 52 in the manner illustrated in FIG. 4.

Another diagnostic device which embodying the present invention is not shown in figures. It includes only one reagent ampoule in the barrel of the swab unit. There is no color developer needed. The second reagent is contained in the outer tube of the cover unit and can be dispensed by puncturing a foil membrane.

As will now be evident to those having ordinary skill in the medical diagnosis arts, informed by the present disclosure, certain embodiments of the present invention may include only one reagent ampoule in the barrel of the swab unit. In some cases the outer tube of the cover unit may contain more than one developer dispensing reagent ampoule. In other cases, no developer reagent is needed and the cover unit may contain second reagent instead. In other cases, the presence of a microorganisms can be determined quantitatively. Other variant embodiments of the present invention by which it is adapted to carry out particular diagnostic procedures will occur to those having ordinary skill in the art without the exercise of invention or undue experimentation.

In the present application, the term “device” and the term “apparatus” are used interchangeably.

In some embodiments of the present invention the reagents employed include (1) a first reagent (“Reagent 1”), which will be contained in ampoule 20, (2) a second reagent (“Reagent 2”), which will be contained in ampoule 22, and (3) a third reagent of color developer (“Developer”), which will be contained in ampoule 42.

In each of the following embodiments where percent solution is not indicated the solution will be a methanol-water solution prepared by dissolving the listed active ingredient, e.g., PYR-MNA in the first embodiment, combining this product with 700 milliliters of distilled water. Where percentage is indicated the solution will be an aqueous solution in the indicated percentage.

In a particular embodiment of the present invention, employing a diagnostic device of the type shown in FIGS. 1 and 2, which is adapted to the detection of Group A streptococcus by swabbing the throat, the three reagents may be:

1. Reagent 1: PYR-MNA (L-Pyrrolidonyl-4-Methoxy-2-Napthylamine) methanol-water solution.

2. Reagent 2: Thyoglycollic acid solution (0.1%).

3. Reagent 3 (Developer): Cinnamaldehyde solution (0.01%).

Given the above-described embodiment of the invention, including the specified reagents, the method of the invention for the detection of Group A streptococcus in the throat may be carried out as follows:

After removing cover unit 14 from swab unit 12, apply swab tip 32 to the throat, and collect on swab tip 32 as much of the specimen material as possible.

Rejoin cover unit 14 and swab unit 12 as shown in FIG. 2

Crush ampoule 20 and then ampoule 22 in the manner indicated in FIG. 3, and hold diagnostic device 10 in a vertical position with swab unit 12 uppermost, so that Reagents 1 and 2 flow, under the influence of gravity, through the hollow interior of shaft 30, and thus permeate swab tip 32, and come into contact with the swab-collected specimen material.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color characteristic of the presence of the microorganism sought to be detected is manifested.

In a particular embodiment of the present invention, employing a diagnostic device of the type shown in FIGS. 1 and 2, which is adapted to the detection of Neisseria Gonorrhea by swabbing the urethra of the cervix of the vagina, the two reagents may be:

Reagent 1: Tetra-alanine-MNA methanol-water solution

Reagent 2 (Developer): Cinnamaldehyde solution (0.01%).

Given the above-described embodiment of the invention, including the specified reagents, the method of the invention for the detection of the specified microorganism or disease, i.e., Neisseria Gonorrhea, may be carried out as follows:

After removing cover unit 14 from swab unit 12, apply swab tip 32 to the body site indicated above, i.e., the urethra or cervix of the vagina, and collect on swab tip 32 as much of the specimen material as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected specimen material.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of the microorganism sought to be detected is manifested.

In a particular embodiment of the present invention, employing a diagnostic device of the type shown in FIGS. 1 and 2, which is adapted to the detection of Urinary tract Infections by treating colleted urine, the three reagents may be:

Reagent 1: Alanine-MNA methanol-water solution.

Reagent 2: Elastase-MNA methanol-water solution.

Reagent 3 (Developer): Cinnamaldehyde solution (0.01%).

Given the above-described embodiment of the invention, including the specified reagents, the method of the invention for the detection of the specified microorganism or disease, may be carried out as follows:

After removing cover unit 14 from swab unit 12, apply swab tip 32 to the urine.

Rejoin cover unit 14 and swab unit 12 as shown in FIG. 2.

Crush ampoule 20 and then ampoule 22 in the manner indicated in FIG. 2, and hold diagnostic device 10 in a vertical position with swab unit 12 uppermost, so that Reagents 1 and 2 flow, under the influence of gravity, through the hollow interior of shaft 30, and thus permeate swab tip 32, and it comes into contact with the swab-collected urine.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of the microorganism sought to be detected is manifested.

In a particular embodiment of the present invention, employing a diagnostic device of the type shown in FIGS. 1 and 2, which is adapted to the detection of Group B streptococcus by swabbing the vaginal area, the two reagents may be:

Reagent 1: Hippurate-MNA methanol-water solution

Reagent 2 (Developer): Cinnamaldehyde solution (0.01%).

Given the above-described embodiment of the invention, including the specified reagents, the method of the invention for the detection of the specified microorganism or disease, i.e., Group B streptococcus may be carried out as follows:

After removing cover unit 14 from swab unit 12, apply swab tip 32 to the vaginal area, and collect on swab tip 32 as much of the discharge found there as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected discharge.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place

After one minute visually examine swab tip 32 through transparent inner tube 34 to determine whether or not the particular unique color (purple) characteristic of the presence of the microorganism sought to be detected is manifested.

In a particular embodiment of the present invention, employing a diagnostic device of the type shown in FIGS. 1 and 2, which is adapted to the detection of Chlamydia by swabbing the genital tract, the two reagents may be:

Reagent 1: SVC-ALA-ALA-Phe-MNA methanol-water solution.

Reagent 2 (Developer): Cinnamaldehyde solution (0.01%).

Given the above-described embodiment of the invention, including the specified reagents, the method of the invention for the detection of Chlamydia may be carried out as follows:

After removing cover unit 14 from swab unit 12, apply swab tip 32 to the genital tract, and collect on swab tip 32 as much of the discharge found there as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected specimen material.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of Chlamydia is manifested.

In a particular embodiment of the present invention, employing a diagnostic device of the type shown in FIGS. 1 and 2, which is adapted to the detection of Pseudomonas Aerginosa by swabbing the infected area, the two reagents may be:

Reagent 1: ABZ-ALA-GLY-LEV-ALA-MNA methanol-water solution.

Reagent 2 (Developer): Cinnamaldehyde solution (0.01%).

Given the above-described embodiment of the invention, including the specified reagents, the method of the invention for the detection of the above-specified microorganism may be carried out as follows:

After removing cover unit 14 from swab unit 12, apply swab tip 32 to the suspected area, and collect on swab tip 32 as much of the discharge found there as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected discharge.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of microorganisms sought to be detected is manifested.

In a particular embodiment of the present invention, employing a diagnostic device of the type shown in FIGS. 1 and 2, which is adapted to the detection of Staphylococcus Aureus by swabbing the suspected area, the two reagents may be:

Reagent 1: N-tosyl-glycyl-L-prolyl-L-arginyl-MNA methanol-water solution.

Reagent 2 (Developer): Cinnamaldehyde solution (0.01%).

Given the above-described embodiment of the invention, including the specified reagents, the method of the invention for the detection of the above-specified microorganism may be carried out as follows:

After removing cover unit 14 from swab unit 12, apply swab tip 32 to the suspected area, and collect on swab tip 32 as much of the discharge found there as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected discharge.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of microorganisms sought to be detected is manifested.

In a particular embodiment of the present invention, employing a diagnostic device of the type shown in FIGS. 1 and 2, which is adapted to the detection of Yeast Infections by swabbing the genital area, the three reagents may be:

Reagent 1: Alanine-MNA methanol-water solution.

Reagent 2: Lysine-Arginine-MNA methanol-water solution.

Reagent 3 (Developer): Cinnamaldehyde solution (0.01%).

Given the above-described embodiment of the invention, including the specified reagents, the method of the invention for the detection of yeast may be carried out as follows:

After removing cover unit 14 from swab unit 12, apply swab tip 32 to the genital area, and collect on swab tip 32 as much of the discharge found there as possible.

Rejoin cover unit 14 and swab unit 12 as shown in FIG. 2.

Crush ampoule 2o and then ampoule 22 in the manner indicated in FIG. 3, and hold diagnostic device 10 in a vertical position with swab unit 12 uppermost, so that Reagents 1 and 2 flows, under the influence of gravity, through the hollow interior of shaft 30, and thus permeate swab tip 32, and it comes into contact with the swab-collected discharge.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of yeast is manifested.

In a particular embodiment of the present invention, employing a diagnostic device of the type shown in FIGS. 1 and 2, which is adapted to the detection of Gram Negative Microorganisms by swabbing the suspected area, the two reagents may be:

Reagent 1: Alanine-MNA methanol-water solution.

Reagent 2 (Developer): Cinnamaldehyde solution (0.01%).

Given the above-described embodiment of the invention, including the specified reagents, the method of the invention for the detection of the specified microorganisms of disease may be carried out as follows:

After removing cover unit 14 from swab unit 12, apply swab tip 32 to the suspected area, and collect on swab tip 32 as much of the discharge found there as possible.

Rejoin cover unit 14 and swab unit 12 as shown in FIG. 2.

Crush ampoule 20 in the manner indicated in FIG. 3, and hold diagnostic device 10 in a vertical position with swab unit 12 uppermost, so that Reagent 1 flows, under the influence of gravity, through the hollow interior of shaft 30, and thus permeates swab tip 32, and it comes into contact with the swab-collected discharge.

After 10 to 15 minutes, crush ampoule 42 by squeezing outer tube 36, and then invert diagnostic device 10, such that plug 18 is at its lower end, and maintain this position while the Developer in ampoule 42 saturates swab tip 32, and the expected color reaction does or does not take place.

After one minute visually examine swab tip 32 through transparent inner tube 34 to determine whether or not the particular unique color (purple) characteristic of the presence of the Gram Negative Microorganisms sought to be detected is manifested.

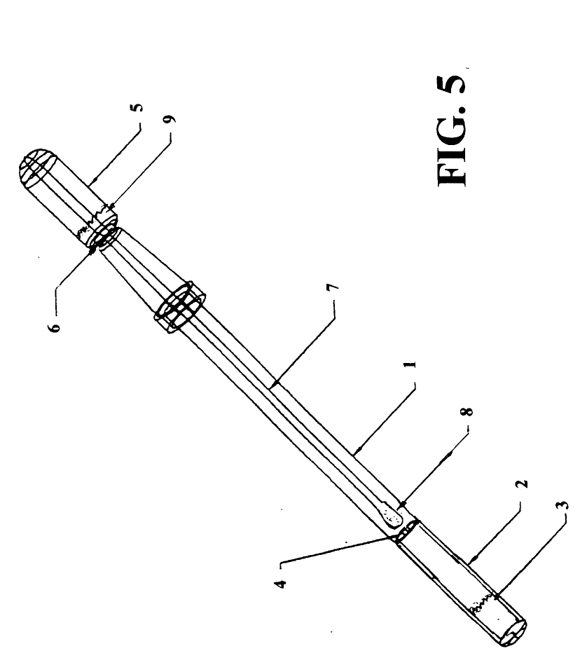

In a particular embodiment of the present invention, employing a diagnostic device of the type shown in FIG. 5, which is adapted to the detection of specific microorganisms by swabbing the testing object, the reagents may be: a buffer solution with a pH range selected from about 6.9 to about 8.7; and one or more reagents selected from the group consisting of the following substrates:

2Hbr-L-Lys AMC
Z-Arg-Arg-AMC
Z-Phe-Pro-Arg-Hbr-AMC
Z-Ala-Ala-Arg-Hbr-AMC
Boc-Leu-Gly-Arg-Hbr-AMC
L-Pro-AMC
(Z)D-Val-Leu-Arg-AMC TFA
D-Ser-Pro-Phe-Arg-AMC 2Hbr
L-Ala-AMC
MeoSuc-Ala-Ala-Pro-Ala-AMC
L-Lys-Lys-Arg-AMC
Z-Leu-Arg-AMC
B-D-N-Acetyl-Glucosaminide-HMC
Z-Gly-Pro-Arg-AMC
L-Pyr-AMC
ABZ-Ala-Gly-Lev-Ala-AMC
N-Tosyl-Glycyl-L-Prolyl-L Arginyl-AMC
L-Ala-Ala-Ala-Ala-AMC
Oleate-HMC
L-Lysyl-Ala-Amc

Given the above-described embodiment of the invention, the method of the invention for the detection of specific microorganisms may be carried out as follows:

A sample is taken from a testing object using a swab 7 that is provided as part of the device.

The swab is then re-introduced into a plastic sleeve 1 of the device tightly.

The snap valve 6 inside a non-rigid barrel 5 is broken and the barrel is gently squeezed to allow the first reagent 9 to moisten the tip of the swab 8.

After 10-15 minutes, holding the two ends of the device, gently push the ends together to push the swab tip 8 through a foil membrane 4. Moisten the swab tip with the second reagent 3 in reagent chamber 2.

After 1-10 minutes, visually examine swab tip 8 for a unique color to determine the presence of a specific microorganism.

It is to be understood that the particular combinations of reagents disclosed herein are not limited in their utility to use in connection with any particular apparatus disclosed herein.

Further, it is to be understood that certain combinations of reagents disclosed herein may be used in other apparatus than those in connection with which they are disclosed herein. Thus, certain combinations of reagents disclosed herein in connection with the embodiment of FIGS. 1 through 3 may also be used in combination with the embodiment of FIG. 4.

It is further to understood that, in general, the combinations of reagents disclosed herein will in most cases find utility in combination with widely available dispensing means, such as reagent tubes, eye dropper bottles, and the like.

The present invention has been described with a certain degree of particularity. It is understood to those skilled in the art that the present disclosure of embodiments has been made by way of examples only and that numerous changes in the arrangement and combination of parts may be resorted without departing from the spirit and scope of the invention. While the embodiments discussed herein may appear to include some limitations as to the presentation of the information units, in terms of the format and arrangement, the invention has applicability well beyond such embodiment, which can be appreciated by those skilled in the art.

1. A method for detecting a microorganism on a testing object, comprising the steps of:
applying a swab to said testing object to collect on said swab a sample from said testing object;
contacting said swab and said sample collected thereon with at least one reagent, wherein said at least one reagent being capable of reacting with said microorganism in said sample to produce a detectable product; and
determining from said detectable product, the presence of said microorganism on said testing object.

2. The method of claim 1 wherein said testing object is a human body site and said sample is a human specimen.

3. The method of claim 1 wherein said testing object is a food surface.

4. The method of claim 1 wherein said detectable product is uniquely fluorescent.

5. The method of claim 1 wherein said detectable product has a unique color.

6. The method of claim 1 wherein said microorganisms is Chlamydia trachomatis, Neisseria gonohorrea, Candida albicans (yeast), Group A Streptococcus, Group B Streptococcus, Cryptosporidium, Staphylococcus aureus or Pseudomonas aerginosa.

7. The method of claim 1 wherein said at least one reagent further comprising:
a first reagent selected from a buffer solution; and
a second reagent selected from the group consisting of the following substrates:
2Hbr-L-Lys AMC
Z-Arg-Arg-AMC
Z-Phe-Pro-Arg-Hbr-AMC
Z-Ala-Ala-Arg-Hbr-AMC
Boc-Leu-Gly-Arg-Hbr-AMC
L-Pro-AMC
(Z)D-Val-Leu-Arg-AMC TFA
D-Ser-Pro-Phe-Arg-AMC 2Hbr
L-Ala-AMC
MeoSuc-Ala-Ala-Pro-Ala-AMC
L-Lys-Lys-Arg-AMC
Z-Leu-Arg-AMC
B-D-N-Acetyl-Glucosaminide-HMC
Z-Gly-Pro-Arg-AMC
L-Pyr-AMC
ABZ-Ala-Gly-Lev-Ala-AMC
N-Tosyl-Glycyl-L-Prolyl-L Arginyl-AMC
L-Ala-Ala-Ala-Ala-AMC
Oleate-HMC
L-Lysyl-Ala-Amc

8. The method of claim 7, wherein said buffer solution has a pH range selected from about 6.9 to about 8.7.

9. The method of claim 7, wherein said at least one reagent further comprising a third reagent selected from a color developer.

10. The method of claim 9, wherein said color developer is an aldehyde.

11. A diagnostic apparatus for detecting a microorganism on a testing object, comprising:
a swab for collecting a sample from a testing object;
means for contacting said swab and said sample collected thereon with at least one reagent, wherein said at least one reagent being capable of reacting with said microorganism in said sample to produce a detectable product; and
means for determining from said detectable product, the presence of said microorganism on said testing object.

12. The apparatus of claim 11 wherein said testing object is a human body site and said sample is a human specimen.

13. The apparatus of claim 11 wherein said testing object is a food surface.

14. The apparatus of claim 11 wherein said detectable product is uniquely fluorescent.

15. The apparatus of claim 11 wherein said detectable product has a unique color.

16. The apparatus of claim 11 wherein said microorganisms is Chlamydia trachomatis, Neisseria gonohorrea, Candida albicans (yeast), Group A Streptococcus, Group B Streptococcus, Cryptosporidium, Staphylococcus aureus or Pseudomonas aerginosa.

17. The apparatus of claim 11 wherein said at least one reagent further comprising:
a first reagent selected from a buffer solution; and
a second reagent selected from the group consisting of the following substrates:
2Hbr-L-Lys AMC
Z-Arg-Arg-AMC
Z-Phe-Pro-Arg-Hbr-AMC
Z-Ala-Ala-Arg-Hbr-AMC
Boc-Leu-Gly-Arg-Hbr-AMC
L-Pro-AMC
(Z)D-Val-Leu-Arg-AMC TFA
D-Ser-Pro-Phe-Arg-AMC 2Hbr
L-Ala-AMC
MeoSuc-Ala-Ala-Pro-Ala-AMC
L-Lys-Lys-Arg-AMC
Z-Leu-Arg-AMC
B-D-N-Acetyl-Glucosaminide-HMC
Z-Gly-Pro-Arg-AMC
L-Pyr-AMC
ABZ-Ala-Gly-Lev-Ala-AMC
N-Tosyl-Glycyl-L-Prolyl-L Arginyl-AMC
L-Ala-Ala-Ala-Ala-AMC
Oleate-HMC
L-Lysyl-Ala-Amc

18. The apparatus of claim 17, wherein said buffer solution has a pH range selected from about 6.9 to about 8.7.

19. The apparatus of claim 17, wherein said at least one reagent further comprising a third reagent selected from a color developer.

20. The apparatus of claim 19, wherein said color developer is an aldehyde.

21. Reagents for detecting a microorganism on a testing object, comprising:
a first reagent selected from a buffer solution; and
a second reagent selected from the group consisting of the following substrates:
2Hbr-L-Lys AMC
Z-Arg-Arg-AMC
Z-Phe-Pro-Arg-Hbr-AMC
Z-Ala-Ala-Arg-Hbr-AMC
Boc-Leu-Gly-Arg-Hbr-AMC
L-Pro-AMC
(Z)D-Val-Leu-Arg-AMC TFA
D-Ser-Pro-Phe-Arg-AMC 2Hbr
L-Ala-AMC
MeoSuc-Ala-Ala-Pro-Ala-AMC
L-Lys-Lys-Arg-AMC
Z-Leu-Arg-AMC
B-D-N-Acetyl-Glucosaminide-HMC
Z-Gly-Pro-Arg-AMC
L-Pyr-AMC
ABZ-Ala-Gly-Lev-Ala-AMC
N-Tosyl-Glycyl-L-Prolyl-L Arginyl-AMC
L-Ala-Ala-Ala-Ala-AMC
Oleate-HMC
L-Lysyl-Ala-Amc

22. The reagents of claim 21, wherein said buffer solution has a pH range selected from about 6.9 to about 8.7.

23. The reagents of claim 21, further comprising a third reagent selected from a color developer.

24. The reagents of claim 23, wherein said color developer is an aldehyde.
